(19) United States
(12) Patent Application Publication
(10) Pub. No.: US 2025/0050062 A1
(43) Pub. Date: Feb. 13, 2025

(54) CATHETER

(71) Applicant: TERUMO KABUSHIKI KAISHA, Tokyo (JP)

(72) Inventors: Daisuke SHIMADA, Fuji-shi (JP); Shintaro FUJINAWA, Hockessin, DE (US); Yuji MOTOSE, Hiratsuka-shi (JP)

(73) Assignee: TERUMO KABUSHIKI KAISHA, Tokyo (JP)

(21) Appl. No.: 18/925,641

(22) Filed: Oct. 24, 2024

Related U.S. Application Data
(63) Continuation of application No. PCT/JP2023/023167, filed on Jun. 22, 2023.

(30) Foreign Application Priority Data
Jul. 28, 2022 (JP) 2022-120521

(51) Int. Cl. A61M 25/00

(52) U.S. Cl. CPC A61M 25/005; A61M 25/0045

(57) ABSTRACT

A catheter that includes a shaft in which a shaft lumen communicating from a distal end to a proximal end; a hub attached to the proximal end of the shaft; and a reinforcing tube surrounding the shaft, includes an inner peripheral surface in contact with an outer peripheral surface of the shaft and an outer peripheral surface in contact with an inner peripheral surface of the hub, and protrudes to a distal end side with respect to a distal end of the hub, in which at least a part of an inner peripheral surface of the reinforcing tube in the hub is welded to at least a part of the outer peripheral surface of the shaft, and at least a part of an outer peripheral surface of the reinforcing tube in the hub is welded to at least a part of the inner peripheral surface of the hub.

### CROSS-REFERENCES TO RELATED APPLICATIONS

This application is a continuation of International Application No. PCT/JP2023/023167 filed on Jun. 22, 2023, which claims priority to Japanese Patent Application No. 2022-120521 filed on Jul. 28, 2022, the entire content of both of which is incorporated herein by reference.

### TECHNOLOGICAL FIELD

The present disclosure generally relates to a catheter.

### BACKGROUND DISCUSSION

In recent years, intraluminal treatment of a blood vessel or the like using a catheter has been actively performed because surgical invasion is very low. A catheter usually has a shaft having a lumen communicating from a distal end to a proximal end, a hub disposed at the proximal end of the shaft, and a kink preventing component that prevents kinking of the shaft in the vicinity of a connecting portion between the shaft and the hub. Catheters with shafts, hubs and kink preventing components are typically assembled by gluing or fitting the respective components together. Therefore, the assembly work of these components may require the skill of an operator.

International Patent Application Publication No. WO 2021/177434 A describes a catheter in which a shaft and a hub are joined by an electromagnetic wave or the like, but a strain relief that is a kink preventing component of the catheter is separated from the shaft and fitted to the hub.

### SUMMARY

A catheter is disclosed that is capable of effectively suppressing the shaft from coming off from a hub of a catheter having a shaft, a hub, and a kink preventing component.

(1) A catheter according to the present disclosure includes: a shaft in which a shaft lumen communicating from a distal end to a proximal end is formed; a hub attached to a proximal end of the shaft; and a reinforcing tube that is a tubular body surrounding the shaft, the reinforcing tube includes an inner peripheral surface in contact with an outer peripheral surface of the shaft and an outer peripheral surface in contact with an inner peripheral surface of the hub, and the reinforcing tube protrudes to a distal end side with respect to a distal end of the hub, in which at least a part of an inner peripheral surface of the reinforcing tube in the hub is welded to at least a part of the outer peripheral surface of the shaft without a gap in a circumferential direction, and at least a part of an outer peripheral surface of the reinforcing tube in the hub is welded to at least a part of the inner peripheral surface of the hub without a gap in a circumferential direction.

In the catheter described in (1), since the shaft, the reinforcing tube, and the hub are welded and integrated, it is possible to effectively suppress the shaft from coming out from the hub of the catheter having the shaft and the reinforcing tube functioning as the hub and the kink preventing component.

(2) In the catheter according to (1), a proximal end of the reinforcing tube may be disposed on a proximal end side with respect to the distal end of the hub and on a distal end side with respect to the proximal end of the shaft, and the outer peripheral surface of a shaft proximal portion of the shaft located on a proximal end side with respect to the proximal end of the reinforcing tube may be welded to an inner peripheral surface of the hub without a gap in a circumferential direction. As a result, since the shaft is not only welded to the hub via the reinforcing tube but also directly welded to the hub, it is possible to effectively prevent the shaft from coming off from the hub.

(3) In the catheter according to (1) or (2), a welded portion to which at least two of the shaft, the hub, or the reinforcing tube are welded may be disposed at least at one location along an axial direction of the shaft. As a result, the catheter can effectively maintain airtightness of the shaft lumen by the welded portion.

(4) In the catheter according to any one of (1) to (3), at least a part of an outer peripheral surface of a shaft reinforcement target portion covered with the reinforcing tube of the shaft may have an uneven surface formed with a surface roughness larger than a surface roughness of the outer peripheral surface of the shaft proximal portion located on a proximal end side with respect to the proximal end of the reinforcing tube of the shaft and brought into close contact with an inner peripheral surface of the reinforcing tube. As a result, since the uneven surface can exhibit an anchor effect of suppressing relative movement between the welded structures, the shaft and the reinforcing tube are more firmly joined to the uneven surface.

(5) In the catheter according to any one of (1) to (4), the reinforcing tube may have a tubular non-welded portion slidable on an outer peripheral surface of the shaft in a range from the distal end to the proximal end side by a predetermined distance. As a result, since the reinforcing tube can slide on the shaft at the tubular non-welded portion, kink of the shaft can be suppressed without hindering flexibility of the shaft.

(6) In the catheter according to any one of (1) to (5), at least a part of the shaft may be formed of a material containing at least one of a polyamide resin or a polyamide elastomer and containing an X-ray contrast material and a pigment, at least a part of the reinforcing tube may be formed of a material containing at least one of a polyamide resin or a polyamide elastomer and containing a pigment, and at least a part of the hub may be formed of a material containing at least one of a polyamide resin, a polyurethane resin, a polyamide elastomer, or a polyurethane elastomer. As a result, the catheter can effectively prevent the shaft from coming out from the hub of the catheter by welding at least two of the shaft, the hub, or the reinforcing tube having the radiopacity.

(7) A catheter according to the present disclosure includes: a shaft, the shaft including a shaft lumen extending from a distal end of the shaft to a proximal end of the shaft; a hub attached to the proximal end of the shaft; a reinforcing tube surrounding the shaft, the reinforcing tube including an inner peripheral surface in contact with an outer peripheral surface of the shaft and an outer peripheral surface in contact with an inner peripheral surface of the hub, and the reinforcing tube protrudes to a distal end side with respect to a distal end of the hub; at least a part of an inner peripheral surface of the reinforcing tube in the hub is welded to at least a part of the outer peripheral surface of the shaft; at least a part of an outer peripheral surface of the reinforcing tube in the hub is welded to at least a part of the inner peripheral surface of the hub; a proximal end of the reinforcing tube is disposed on a proximal end side with respect to the distal end of the hub and on a distal end side with respect to the proximal end of the shaft; and the outer peripheral surface of a shaft proximal portion of the shaft located on a proximal end side with respect to the proximal end of the reinforcing tube is welded to an inner peripheral surface of the hub.

(8) A catheter according to the present disclosure includes: an elongated shaft; a hub attached to a proximal end of the shaft; a reinforcing tube surrounding the shaft, the reinforcing tube including an inner peripheral surface in contact with an outer peripheral surface of the shaft and an outer peripheral surface in contact with an inner peripheral surface of the hub, and the reinforcing tube protrudes to a distal end side with respect to a distal end of the hub; at least a part of an inner peripheral surface of the reinforcing tube in the hub is joined to at least a part of the outer peripheral surface of the shaft; at least a part of an outer peripheral surface of the reinforcing tube in the hub is joined to at least a part of the inner peripheral surface of the hub; a proximal end of the reinforcing tube is disposed on a proximal end side with respect to the distal end of the hub and on a distal end side with respect to the proximal end of the shaft; and the outer peripheral surface of a shaft proximal portion of the shaft located on a proximal end side with respect to the proximal end of the reinforcing tube is joined to an inner peripheral surface of the hub.

### BRIEF DESCRIPTION OF THE DRAWINGS

FIG. 1 is a plan view illustrating a catheter according to an embodiment.

FIG. 2 is a cross-sectional view illustrating a shaft, a reinforcing tube, and a hub of a catheter.

FIGS. 3A to 3C are views illustrating a catheter, in which FIG. 3A is a cross-sectional view taken along line A-A in FIG. 2, FIG. 3B is a cross-sectional view taken along line B-B in FIG. 2, and FIG. 3C is a cross-sectional view taken along line C-C in FIG. 2.

FIG. 4 is a cross-sectional view illustrating a shaft, a reinforcing tube, and a hub of a catheter according to a first modification.

FIG. 5 is a plan view illustrating a catheter according to a second modification.

FIG. 6 is a plan view illustrating a catheter according to a third modification.

FIG. 7 is a cross-sectional view illustrating a shaft, a reinforcing tube, and a hub of a catheter according to a fourth modification.

### DETAILED DESCRIPTION

Set forth below with reference to the accompanying drawings is a detailed description of embodiments of a catheter. Note that, dimensional ratios in the drawings are sometimes exaggerated and different from actual ratios for convenience of description. In the following description, a side on which a catheter is operated is referred to as a “proximal end side”, and a side to be inserted into a living body is referred to as a “distal end side”.

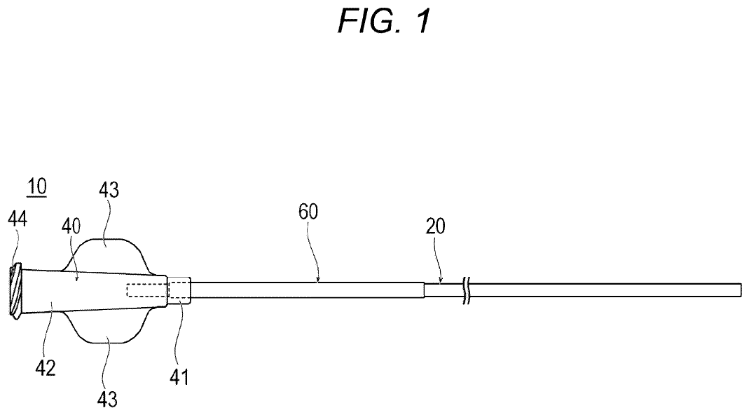

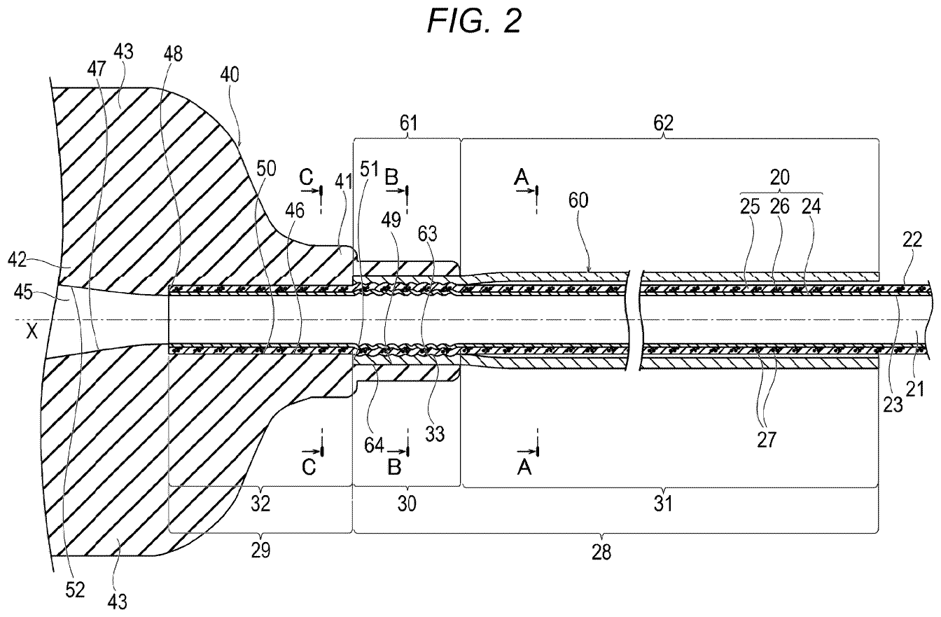

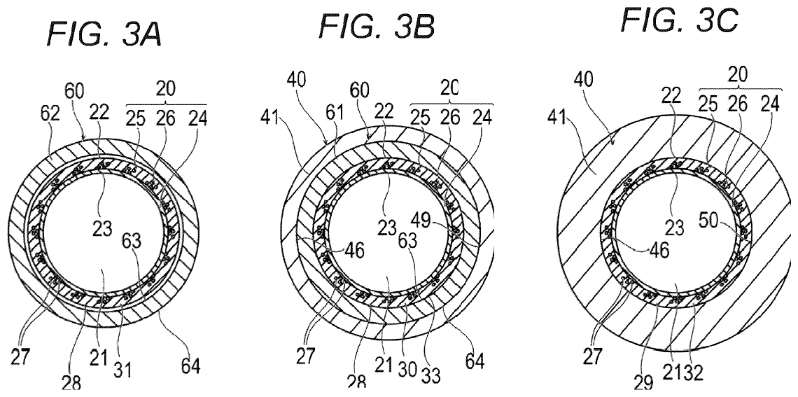

As illustrated in FIGS. 1 to 3C, a catheter 10 according to an embodiment of the present disclosure includes a shaft 20 that is an elongated tubular body, a hub 40 fixed to a proximal end of the shaft 20, and a reinforcing tube 60 that covers the shaft 20 in order to suppress bending of the shaft 20. In addition to a catheter supporting a guide wire, the catheter 10 may be a guiding catheter, an angiographic catheter, a microcatheter, a balloon catheter having a lumen for expansion, or a diagnostic imaging catheter. In addition, the catheter 10 may be of an over-the-wire (OTW) type in which a guide wire lumen communicating from the distal end of the shaft to the hub is formed, or may be of a rapid exchange (RX) type in which a guide wire lumen is formed only at the distal portion of the shaft. For example, a guide wire lumen of an RX type balloon catheter is formed from a distal end of the shaft to an opening in the middle of the shaft in the axial direction. An expansion lumen through which a fluid for expanding a balloon of an RX type balloon catheter flows is formed to communicate from the balloon to a hub at a proximal end of the catheter.

The shaft 20 is a tubular body in which a shaft lumen 21 communicating from the distal end to the proximal end of shaft 20 is formed. The shaft 20 has a shaft outer peripheral surface 22 and a shaft inner peripheral surface 23. The shaft outer peripheral surface 22 is a radially outer surface of the shaft 20 that is a tubular body, and extends from the distal end to the proximal end of the shaft 20. The shaft inner peripheral surface 23 is a radially inner surface of the shaft 20 that is a tubular body, and extends from the distal end to the proximal end of the shaft 20.

The shaft 20 in the present embodiment includes the inner layer 24 forming the shaft inner peripheral surface 23, the outer layer 25 forming the shaft outer peripheral surface 22, and a reinforcing body 26 embedded in the shaft 20, but is not limited to the embodiment described above, and may be a multilayer resin tube having the outer layer 25 and the inner layer 24 not including the reinforcing body 26, a single-layer resin tube, or a metal hypotube coated with a resin outside.

Examples of the constituent material of the outer layer 25 can include, in addition to a polyamide resin, a polyester resin, a polyolefin resin, and a polyurethane resin, a polyamide elastomer, a polyester elastomer, a polyurethane elastomer, or a mixture of two or more of the materials listed above. The outer layer 25 may be formed by arranging materials having different hardness so as to be flexible from the proximal end toward the distal end, but is not limited to the embodiment described.

The constituent material of the inner layer 24 may be the same material as the constituent material of the outer layer 25 described above, or may be a material different from the constituent material of the outer layer 25. The constituent material of the inner layer 24 may be a fluorine-based resin material such as a polytetrafluoroethylene resin in order to enhance the slidability of the shaft inner peripheral surface 23, but is not limited to a fluorine-based resin material such as polytetrafluoroethylene.

The reinforcing body 26 reinforces the shaft 20, and is formed by braiding a plurality of wire rods 27 into a tubular shape. In addition, the reinforcing body 26 may be formed by spirally winding one or more wire rods. The material of the outer layer 25 or the inner layer 24 enters a gap between the plurality of wire rods 27 in the reinforcing body 26. The wire rod 27 is made of metal such as stainless steel or NiTi (nickel-titanium), but is not limited to stainless steel or NiTi, and may be formed by spirally winding one wire rod 27 with a coil, and the reinforcing body 26 may be provided on a part of the shaft 20.

In addition, the shaft 20 includes a shaft reinforcement target portion 28 covered with the reinforcing tube 60 and a shaft proximal portion 29 located on the proximal end side of the shaft reinforcement target portion 28. The shaft proximal portion 29 is disposed on the proximal end side of the proximal end of the reinforcing tube 60 and is not covered with the reinforcing tube 60. The shaft reinforcement target portion 28 includes a first shaft welded portion 30 having the shaft outer peripheral surface 22 to which the inner peripheral surface of the reinforcing tube 60 is welded (i.e., joined by heating), and a shaft non-welded portion 31 having the shaft outer peripheral surface 22 to which the inner peripheral surface of the reinforcing tube 60 is not welded. The shaft proximal portion 29 includes a second shaft welded portion 32 having the shaft outer peripheral surface 22 to which the inner peripheral surface of the hub 40 is welded.

The shaft inner peripheral surface 23 and the shaft outer peripheral surface 22 located in the first shaft welded portion 30 are formed with unevenness (i.e., uneven inner peripheral and outer peripheral surfaces 23, 22). Therefore, the shaft outer peripheral surface 22 located in the first shaft welded portion 30 has the uneven surface 33 formed with a surface roughness larger than that of the shaft outer peripheral surface 22 located in the second shaft welded portion 32. The shaft outer peripheral surface 22 located in the second shaft welded portion 32 is welded to a second hub welded surface 50 described later that is a part of the inner peripheral surface forming a hub lumen 45 of the hub 40, without a gap in the circumferential direction. Therefore, the uneven surface 33 is welded in close contact with the inner peripheral surface of the reinforcing tube 60 without a gap. Note that the state of being in close contact in the circumferential direction is a state in which the entire portion in the circumferential direction over 360 degrees is in close contact with the shaft 20 without a gap in a predetermined cross section orthogonal to the axis center X of the shaft, and airtightness between the distal end side and the proximal end side is maintained beyond the cross section. Since the uneven surface 33 can exhibit an anchor effect of suppressing relative movement between the welded structures, the shaft 20 and the reinforcing tube 60 are more firmly joined to the first shaft welded portion 30 having the uneven surface 33. Note that, when the uneven surface 33 is formed on the shaft outer peripheral surface 22, the first shaft welded portion 30 also has unevenness on the shaft inner peripheral surface 23, but may not have unevenness. Since the unevenness of the shaft inner peripheral surface 23 is not so large as to hinder the movement of the device and the fluid through the shaft lumen 21, there is no problem in the function of the catheter 10. Note that the first shaft welded portion 30 may not have unevenness on the shaft outer peripheral surface 22 and the shaft inner peripheral surface 23, and bubbles or the like may be generated between the shaft outer peripheral surface 22 and the shaft inner peripheral surface 23 as long as airtightness is maintained.

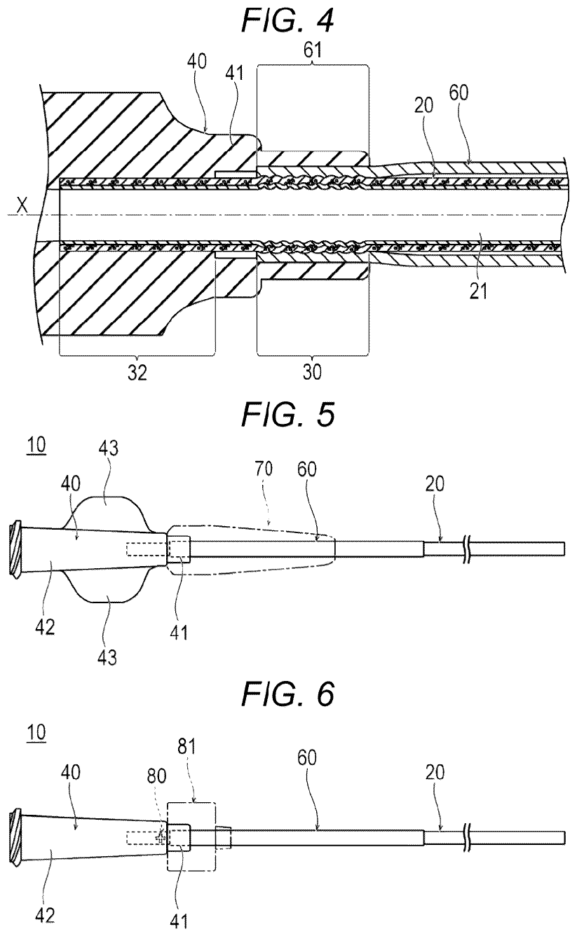

In the present embodiment, the welded portion including the first shaft welded portion 30, the second shaft welded portion 32, and a tubular welded portion 61 is formed without a gap along the axis center X of the shaft 20, but may be formed at a plurality of places separated along the axial direction of the shaft 20. In the first modification illustrated in FIG. 4, a second welded portion 32 is formed separated from a first welded portion 30 and the tubular welded portion 61 in the proximal end direction. The welded portions of the shaft 20 are disposed at two places separated from each other along the axial direction of the shaft 20. When the laser absorptivity of the reinforcing tube 60 and the laser absorptivity of the shaft 20 are greatly different, it can be relatively easy to separately provide the welded portions of the reinforcing tube 60 and the shaft 20. Therefore, the plurality of welded portions can be appropriately formed, and a defect in appearance can be suppressed.

As illustrated in FIGS. 1 to 3, the reinforcing tube 60 is a tubular body that covers the vicinity of the proximal portion of the shaft 20. The distal end of the reinforcing tube 60 is disposed on the distal end side with respect to the distal end of the hub 40, and is disposed on the proximal end side with respect to the distal end of the shaft 20. The proximal end of the reinforcing tube 60 is disposed on the proximal end side with respect to the distal end of the hub 40 and on the distal end side with respect to the proximal end of the shaft 20. The reinforcing tube 60 covers the shaft reinforcement target portion 28 of the shaft 20.

The reinforcing tube 60 includes the tubular welded portion 61 having an inner peripheral surface welded to the shaft outer peripheral surface 22 and a tubular non-welded portion 62 having an inner peripheral surface not welded to the shaft outer peripheral surface 22. In addition, the reinforcing tube 60 has a tubular outer peripheral surface 64 and a tubular inner peripheral surface 63. The tubular outer peripheral surface 64 is a radially outer surface of the reinforcing tube 60 that is a tubular body, and extends from the distal end to the proximal end of the reinforcing tube 60. The tubular inner peripheral surface 63 is a radially inner surface of the reinforcing tube 60 that is a tubular body, and extends from the distal end to the proximal end of the reinforcing tube 60. The tubular inner peripheral surface 63 located in the tube welded portion 61 covers the first shaft welded portion 30 and is welded to the shaft outer peripheral surface 22 located in the first shaft welded portion 30. In addition, the tubular inner peripheral surface 63 located in the tubular non-welded portion 62 covers the shaft non-welded portion 31 through a small gap, and can slidably contact the shaft outer peripheral surface 22 located in the shaft non-welded portion 31. Note that the reinforcing tube 60 may have the tubular welded portion 61 in the entire axial direction and may not have the tubular non-welded portion 62.

The tubular inner peripheral surface 63 located in the tubular welded portion 61 is welded to the shaft outer peripheral surface 22 located in the first shaft welded portion 30 without a gap in the circumferential direction. Since the shaft outer peripheral surface 22 of the first shaft welded portion 30 has the uneven surface 33, the tubular inner peripheral surface 63 located in the tubular welded portion 61 has an uneven shape corresponding to the uneven surface 33 so as to be in close contact with the uneven surface 33. Note that a gap may be partially formed between the tubular inner peripheral surface 63 located in the tubular welded portion 61 and the shaft outer peripheral surface 22 located in the first shaft welded portion 30.

The tubular outer peripheral surface 64 located in the tubular welded portion 61 is welded to a first hub welded surface 49 described later that is a part of the inner peripheral surface forming the hub lumen 45 of the hub 40, without a gap in the circumferential direction.

Examples of the constituent material of the reinforcing tube 60 include materials applicable to the outer layer 25 described above. The material of the reinforcing tube 60 may be the same or different than the material of the outer layer 25.

The hub 40 includes a tubular housing portion 41 located on the distal end side and housing the shaft 20 and the proximal portion of the reinforcing tube 60, a hub body 42 disposed on the proximal end side of the housing portion 41, wings 43, and a thread-cutting protrusion 44. In the hub 40, the hub lumen 45 communicating from the distal end to the proximal end is formed. The hub lumen 45 includes a housing surface 46 that is an inner peripheral surface of the housing portion 41, a hub passage 47 that is an inner peripheral surface of the hub body 42, and a first step 48 between the housing surface 46 and the hub passage 47.

The housing surface 46 includes the first hub welded surface 49 directly welded to the tubular welded portion 61 of the reinforcing tube 60, the second hub welded surface 50 directly welded to the second shaft welded portion 32 of the shaft 20, and a second step 51 between the first hub welded surface 49 and the second hub welded surface 50.

The first hub welded surface 49 extends in the proximal end direction from the distal end of the housing surface 46. The second hub welded surface 50 extends in the distal end direction from the proximal end of the housing surface 46. The second hub welded surface 50 is disposed on the proximal end side of the first hub welded surface 49 with the second step 51 interposed between the second hub welded surface 50 and the proximal end side of the first hub welded surface 49. The inner diameter of the second hub welded surface 50 is smaller than the inner diameter of the first hub welded surface 49. Therefore, the second step 51 having the inner diameter locally changes is formed between the first hub welded surface 49 and the second hub welded surface 50. The second step 51 is an annular surface facing the distal end side, and is formed substantially perpendicular to the axis center X of the shaft 20. The radially outer side of the second step 51 is connected to the first hub welded surface 49. The radially inner side of the second step 51 is connected to the distal end of the second hub welded surface 50. The second step 51 is welded by abutting on the proximal end surface of the reinforcing tube 60, but may not be welded.

The hub passage 47 has a tapered portion 52 having an inner diameter gradually increasing in the proximal end direction from the housing surface 46 and formed in a tapered shape. The inner diameter of the distal end of the hub passage 47 coincides with the inner diameter of the shaft proximal portion 29. Therefore, the inner peripheral surface of the hub passage 47 is continuous from the inner peripheral surface of the shaft proximal portion 29 with a constant inner diameter without a step. A portion of the tapered portion 52 may be a luer taper connectable with a syringe. The guide wire or the treatment catheter 10 inserted from the proximal end opening of the hub lumen 45 can smoothly pass through the hub lumen 45 and the shaft lumen 21 without a step and protrude from the distal end of the catheter 10. As a result, the guide wire and the treatment catheter can rather easily reach a target position such as a lesion.

The first step 48 is an annular surface facing the distal end side, and is formed substantially perpendicular to the axis center X of the shaft 20. The radially outer side of the first step 48 is connected to the second hub welded surface 50. The radially outer side of the first step 48 is connected to the distal end of the hub passage 47. The first step 48 is welded by abutting on the proximal end surface of the shaft 20, but may not be welded. The hub passage 47 is preferably coaxial with the housing surface 46 and further coaxial with the shaft lumen 21.

The wings 43 are formed to protrude from two opposing portions of the outer peripheral surface of the hub body 42 so that the operator can easily grip and operate the hub 40. The thread-cutting protrusion 44 is formed on the outer peripheral surface on the proximal end side of the hub body 42. The thread-cutting protrusion 44 can be engaged with a lock-type syringe or the like. Note that the hub 40 may not include the wings 43 or the thread-cutting protrusion 44.

The constituent material of the hub 40 is not particularly limited as long as it is a thermoplastic resin, but is preferably one that easily transmits heat or electromagnetic waves, and specific examples of materials of the hub 40 can include polyamide resins, polyester resins, polyolefin resins, polyurethane resins, as well as polyamide elastomers, polyester elastomers, polyurethane elastomers, and mixtures of two or more of the materials.

When the shaft 20, the reinforcing tube 60, and the hub 40 are welded, a mandrel, for example, can be is inserted into the shaft lumen 21 and the hub lumen 45, and the reinforcing tube 60 is put on the shaft 20. Then, the shaft 20 and the reinforcing tube 60 are inserted into the housing portion 41 of the hub 40, the proximal end surface of the shaft 20 is abutted against the first step 48 of the hub 40, and the proximal end surface of the reinforcing tube 60 is abutted against the second step 51 of the hub 40. Next, a portion where the shaft 20, the reinforcing tube 60, and the hub 40 are welded is heated. As a result, the first hub welded surface 49 of the hub 40 and the tubular outer peripheral surface 64 located at the tubular welded portion 61 of the reinforcing tube 60 are welded, and the second hub welded surface 50 of the hub 40 and the shaft outer peripheral surface 22 located at the shaft proximal portion 29 of the shaft 20 are welded. Further, the tubular inner peripheral surface 63 located at the tubular welded portion 61 of the reinforcing tube 60 and the shaft outer peripheral surface 22 located at the second shaft welded portion 32 of the shaft 20 are welded to each other. Each welded portion may have an integrated structure by mixing materials. Note that the heating method is not particularly limited, and examples of the heating method can include a method of irradiating with an electromagnetic wave having a wavelength that is transmitted through the hub 40, partially transmitted through the reinforcing tube 60, and not transmitted (or only partially transmitted) through the shaft 20. As a result, the tubular welded portion 61, the first shaft welded portion 30, and the second shaft welded portion 32 are heated by the electromagnetic wave and partially melted. Then, the heat of the shaft 20 and the reinforcing tube 60 is transmitted to the housing portion 41 of the hub 40, and the first hub welded surface 49 and the second hub welded surface 50 of the housing portion 41 are melted.

The electromagnetic wave can include heat, microwaves, visible light, and infrared rays. The electromagnetic wave may be a laser. The infrared ray is a near-infrared ray having a wavelength, for example, of about 0.7 μm to 2.5 μm, a mid-infrared ray having a wavelength, for example, of about 2.5 μm to 4 μm, or a far-infrared ray having a wavelength, for example, of about 4 μm to 1000 μm, but may include only one or two or more kinds of near-infrared rays, mid-infrared rays, or far-infrared rays, or may include visible light or a microwave.

The outer layer 25 of the shaft 20 and the reinforcing tube 60 may be mixed with a pigment that does not transmit or absorbs heat or electromagnetic waves so as to be heated by irradiation with electromagnetic waves. Alternatively, the outer layer 25 and the reinforcing tube 60 may not contain a pigment, and the resin forming the outer layer 25 and the reinforcing tube 60 may have low transmittance for a specific wavelength. In addition, the outer layer 25 is preferably mixed with a material having radiopacity such as barium sulfate, but may not be mixed. In addition, usually, a material having radiopacity such as barium sulfate is not mixed with the reinforcing tube 60, but may be mixed with the reinforcing tube 60.

The pigment is not particularly limited as long as it is a pigment that develops white, black, blue, red, or yellow, or a mixture of white, black, blue, red or yellow, but in the present embodiment, a pigment that develops a color other than white or black is preferable.

In addition, the catheter 10 may have a strain relief 70 having a structure different from that of the reinforcing tube 60 as in the second modification illustrated in FIG. 5. The strain relief 70 further suppresses the kink of the shaft 20 together with the reinforcing tube 60 by surrounding the connecting portion between the hub 40 and the reinforcing tube 60. The strain relief 70 is fixed by being screwed to a screw thread formed on the outer peripheral surface of the housing portion 41 of the hub 40 or by being adhered to the outer peripheral surface of the housing portion 41 or the reinforcing tube 60.

In addition, in the catheter 10, as in the third modification illustrated in FIG. 6, a visual marker 80 that can be visually recognized through the transparent hub 40 may be disposed between the hub 40 and the shaft 20 or between the hub 40 and the reinforcing tube 60. The visual marker 80 is disposed at a specific position in the circumferential direction. The catheter 10 can be, for example, an inner catheter or a guide wire integrated catheter that is used by being inserted into the outer catheter from the proximal end side, has a lock adapter 81 that can be fixed to the outer catheter, and does not have wings. In this case, when the catheter 10 is inserted into the outer catheter and fixed to the outer catheter by the lock adapter 81, the operator can fix the catheter 10 to the outer catheter at an appropriate angle with respect to the outer catheter 10 even if the wing cannot be used as the marker by visually recognizing the visual marker 80.

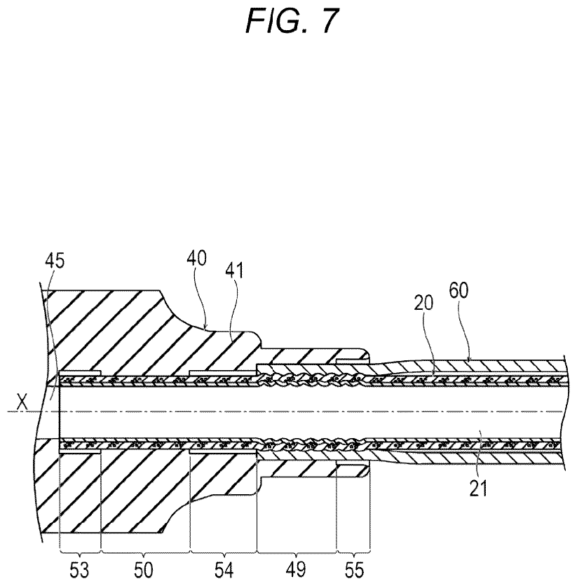

In addition, as in the fourth modification illustrated in FIG. 7, the hub 40 may have a first non-welded portion 53 disposed with a gap interposed between the first non-welded portion 53 and the outer peripheral surface of the shaft 20 on the inner peripheral surface within a predetermined range from the most proximal end toward the distal end side of the shaft 20 and not welded to the outer peripheral surface of the shaft 20. In addition, the hub 40 may have a second non-welded portion 54 disposed with a gap interposed between the second non-welded portion 54 and the outer peripheral surface of the shaft 20 on the inner peripheral surface within a predetermined range from the most proximal end toward the reinforcing tube 60 and not welded to the outer peripheral surface of the shaft 20. The first non-welded portion 53 is located on the proximal end side of the second hub welded surface 50 adjacent to the second hub welded surface 50 welded to the outer peripheral surface of the shaft 20, and the second non-welded portion 54 is located on the distal end side of the second hub welded surface 50 adjacent to the second hub welded surface 50. In addition, the hub 40 may have a third non-welded portion 55 that is disposed with a gap interposed between the third non-welded portion 55 and the outer peripheral surface of the reinforcing tube 60 and is not welded to the outer peripheral surface of the reinforcing tube 60 in a predetermined range from the most distal end toward the proximal end side. The third non-welded portion 55 is located on the distal end side of the first hub welded surface 49 adjacent to the first hub welded surface 49 welded to the outer peripheral surface of the reinforcing tube 60. The third non-welded portion 55 is open to the outside on the distal end side.

The inner diameters of the first non-welded portion 53 and the second non-welded portion 54 coincide with the inner diameter of the second hub welded surface 50 before welding processing, but are formed larger than the inner diameter of the second hub welded surface 50 by reducing the diameter of the second hub welded surface 50 during welding processing. By forming the first non-welded portion 53 on the hub 40, it is possible to prevent the tapered portion 52 of the hub 40 on the proximal end side of the shaft 20 from being heated and deformed at the time of welding processing. As a result, it is possible to suppress the guide wire from being rather easily caught in the hub lumen 45. In addition, since the second non-welded portion 54 is formed in the hub 40, it is possible to suppress the hub 40 from being heated and deformed in the vicinity of the proximal end of the reinforcing tube 60 at the time of welding.

The inner diameter of the third non-welded portion 55 matches the inner diameter of the first hub welded surface 49 before welding processing, but is formed larger than the inner diameter of the first hub welded surface 49 by reducing the diameter of the first hub welded surface 49 during welding processing. Since the third non-welded portion 55 is formed in the hub 40, it is possible to suppress the vicinity of the distal end of the hub 40 from being heated and deformed at the time of welding processing. By suppressing deformation in the vicinity of the distal end of the hub 40, the strain relief 70 can be relatively easily connected to the distal portion of the hub 40. Further, the inner diameter of the opening at the most distal end of the third non-welded portion 55 may be larger than the proximal end side of the third non-welded portion 55 or may be enlarged in a reverse tapered shape.

The first non-welded portion 53, the second non-welded portion 54, and the third non-welded portion 55 are formed within a range in which there is no problem in pressure resistance and liquid leakage of the catheter 10. Note that the gaps in the first non-welded portion 53, the second non-welded portion 54, and the third non-welded portion 55 are different from, for example, air bubbles and voids formed due to insufficient inflow of the adhesive.

As described above, the catheter 10 of the aspect (1) according to the present embodiment includes: a shaft 20 in which a shaft lumen 21 communicating from a distal end to a proximal end is formed; a hub 40 attached to a proximal end of the shaft 20; and a reinforcing tube 60 that is a tubular body surrounding the shaft 20, the reinforcing tube 60 includes an inner peripheral surface in contact with an outer peripheral surface of the shaft 20 and an outer peripheral surface in contact with an inner peripheral surface of the hub 40, and protrudes to a distal end side with respect to a distal end of the hub 40, in which at least a part of an inner peripheral surface of the reinforcing tube 60 in the hub 40 is welded to at least a part of the outer peripheral surface of the shaft 20 without a gap in a circumferential direction, and at least a part of an outer peripheral surface of the reinforcing tube 60 in the hub 40 is welded to at least a part of the inner peripheral surface of the hub 40 without a gap in a circumferential direction.

As a result, in the catheter 10 of the aspect (1), since the shaft 20, the reinforcing tube 60, and the hub 40 are welded and integrated, it is possible to effectively suppress the shaft 20 from coming out from the hub 40 of the catheter 10 having the shaft 20 and the reinforcing tube 60 functioning as the hub 40 and the kink preventing component.

Regarding the catheter 10 of the aspect (2), in the catheter 10 of the aspect (1), a proximal end of the reinforcing tube 60 is disposed on a proximal end side with respect to the distal end of the hub 40 and on a distal end side with respect to the proximal end of the shaft 20, and the outer peripheral surface of a shaft proximal portion 29 of the shaft 20 located on a proximal end side with respect to the proximal end of the reinforcing tube 60 is welded to an inner peripheral surface of the hub 40 without a gap in a circumferential direction. As a result, since the shaft 20 is not only welded to the hub 40 via the reinforcing tube 60 but also directly welded to the hub 40, it is possible to effectively prevent the shaft 20 from coming off from the hub 40.

Regarding the catheter 10 of the aspect (3), in the catheter 10 of the aspect (1) or (2), the welded portion (the first shaft welded portion 30, the second shaft welded portion 32 or the tubular welded portion 61) where at least two of the shaft 20, the hub 40, or the reinforcing tube 60 are welded is disposed at least at one location along the axial direction of the shaft 20. As a result, the catheter 10 can effectively maintain airtightness of the shaft lumen 21 by the welded portion.

Regarding the catheter 10 of the aspect (4), in the catheter 10 of any one of the aspects (1) to (3), at least a part of an outer peripheral surface of a shaft reinforcement target portion 28 covered with the reinforcing tube 60 of the shaft 20 has an uneven surface 33 formed with a surface roughness larger than a surface roughness of the outer peripheral surface of the shaft proximal portion 29 located on a proximal end side with respect to the proximal end of the reinforcing tube 60 of the shaft 20 and brought into close contact with an inner peripheral surface of the reinforcing tube 60. As a result, since the uneven surface 33 can exhibit an anchor effect of suppressing relative movement between the welded structures, the shaft 20 and the reinforcing tube 60 are more firmly joined to the uneven surface 33.

Regarding the catheter 10 of the aspect (5), in the catheter 10 of any one of the aspects (1) to (4), the reinforcing tube 60 has a tubular non-welded portion 62 slidable on an outer peripheral surface of the shaft 20 in a range from the distal end to the proximal end side by a predetermined distance. As a result, since the tubular non-welded portion 62 of the reinforcing tube 60 can slide on the shaft 20 at the tubular non-welded portion 62, kink of the shaft 20 can be suppressed without hindering flexibility of the shaft 20.

Regarding the catheter 10 of the aspect (6), in the catheter 10 of any one of aspects (1) to (5), at least a part of the shaft 20 is formed of a material containing at least one of a polyamide resin or a polyamide elastomer and containing an X-ray contrast material and a pigment, at least a part of the reinforcing tube 60 is formed of a material containing at least one of a polyamide resin or a polyamide elastomer and containing a pigment, and at least a part of the hub 40 is formed of a material containing at least one of a polyamide resin, a polyurethane resin, a polyamide elastomer, or a polyurethane elastomer. As a result, the catheter 10 can effectively prevent the shaft 20 from coming out from the hub 40 of the catheter 10 by welding at least two of the shaft 20, the hub 40, or the reinforcing tube 60 having the radiopacity.

Note that, the present disclosure is not limited to the embodiment described above, and various modifications may be made by those skilled in the art within the technical idea of the present invention. For example, the shaft 20 may be heated by high-frequency induction heating that uses electromagnetic induction. The electromagnetically induced electrical conductor is, for example, a reinforcing body 26.

The detailed description above describes embodiments of a catheter. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A catheter comprising:
a shaft in which a shaft lumen communicating from a distal end to a proximal end is formed;
a hub attached to the proximal end of the shaft;
a reinforcing tube that is a tubular body surrounding the shaft, the reinforcing tube includes an inner peripheral surface in contact with an outer peripheral surface of the shaft and an outer peripheral surface in contact with an inner peripheral surface of the hub, and the reinforcing tube protrudes to a distal end side with respect to a distal end of the hub;
at least a part of an inner peripheral surface of the reinforcing tube in the hub is welded to at least a part of the outer peripheral surface of the shaft without a gap in a circumferential direction; and
at least a part of an outer peripheral surface of the reinforcing tube in the hub is welded to at least a part of the inner peripheral surface of the hub without a gap in a circumferential direction.

2. The catheter according to claim 1, wherein
a proximal end of the reinforcing tube is disposed on a proximal end side with respect to the distal end of the hub and on a distal end side with respect to the proximal end of the shaft; and
the outer peripheral surface of a shaft proximal portion of the shaft located on a proximal end side with respect to the proximal end of the reinforcing tube is welded to an inner peripheral surface of the hub without a gap in a circumferential direction.

3. The catheter according to claim 1, wherein a welded portion to which at least two of the shaft, the hub, or the reinforcing tube are welded is disposed at least at one location along an axial direction of the shaft.

4. The catheter according to claim 1, wherein at least a part of an outer peripheral surface of a shaft reinforcement target portion covered with the reinforcing tube of the shaft has an uneven surface formed with a surface roughness larger than a surface roughness of the outer peripheral surface of the shaft proximal portion located on a proximal end side with respect to the proximal end of the reinforcing tube of the shaft and brought into close contact with an inner peripheral surface of the reinforcing tube.

5. The catheter according to claim 1, wherein the reinforcing tube has a tubular non-welded portion slidable on an outer peripheral surface of the shaft in a range from the distal end to the proximal end side by a predetermined distance.

6. The catheter according to claim 1, wherein
at least a part of the shaft is formed of a material containing at least one of a polyamide resin or a polyamide elastomer and containing an X-ray contrast material and a pigment;
at least a part of the reinforcing tube is formed of a material containing at least one of a polyamide resin or a polyamide elastomer and containing the pigment; and
at least a part of the hub is formed of a material containing at least one of a polyamide resin, a polyurethane resin, a polyamide elastomer, or a polyurethane elastomer.

7. A catheter comprising:
a shaft, the shaft including a shaft lumen extending from a distal end of the shaft to a proximal end of the shaft;
a hub attached to the proximal end of the shaft;
a reinforcing tube surrounding the shaft, the reinforcing tube including an inner peripheral surface in contact with an outer peripheral surface of the shaft and an outer peripheral surface in contact with an inner peripheral surface of the hub, and the reinforcing tube protrudes to a distal end side with respect to a distal end of the hub;
at least a part of an inner peripheral surface of the reinforcing tube in the hub is welded to at least a part of the outer peripheral surface of the shaft;
at least a part of an outer peripheral surface of the reinforcing tube in the hub is welded to at least a part of the inner peripheral surface of the hub;
a proximal end of the reinforcing tube is disposed on a proximal end side with respect to the distal end of the hub and on a distal end side with respect to the proximal end of the shaft; and
the outer peripheral surface of a shaft proximal portion of the shaft located on a proximal end side with respect to the proximal end of the reinforcing tube is welded to an inner peripheral surface of the hub.

8. The catheter according to claim 7, wherein a welded portion to which at least two of the shaft, the hub, or the reinforcing tube are welded is disposed at least at one location along an axial direction of the shaft.

9. The catheter according to claim 7, wherein at least a part of an outer peripheral surface of a shaft reinforcement target portion covered with the reinforcing tube of the shaft has an uneven surface formed with a surface roughness larger than a surface roughness of the outer peripheral surface of the shaft proximal portion located on a proximal end side with respect to the proximal end of the reinforcing tube of the shaft and brought into close contact with an inner peripheral surface of the reinforcing tube.

10. The catheter according to claim 7, wherein the reinforcing tube has a tubular non-welded portion slidable on an outer peripheral surface of the shaft in a range from the distal end to the proximal end side by a predetermined distance.

11. The catheter according to claim 7, wherein
at least a part of the shaft is formed of a material containing at least one of a polyamide resin or a polyamide elastomer and containing an X-ray contrast material and a pigment.

12. The catheter according to claim 7, wherein
at least a part of the reinforcing tube is formed of a material containing at least one of a polyamide resin or a polyamide elastomer and containing a pigment.

13. The catheter according to claim 7, wherein
at least a part of the hub is formed of a material containing at least one of a polyamide resin, a polyurethane resin, a polyamide elastomer, or a polyurethane elastomer.

14. The catheter according to claim 7, wherein an outer layer of the shaft and the reinforcing tube are mixed with a pigment that does not transmit or absorb heat or electromagnetic waves.

15. The catheter according to claim 7, wherein an outer layer and the reinforcing tube are formed from a resin having a low transmittance to a wavelength of electromagnetic waves.

16. A catheter comprising:
an elongated shaft;
a hub attached to a proximal end of the shaft;
a reinforcing tube surrounding the shaft, the reinforcing tube including an inner peripheral surface in contact with an outer peripheral surface of the shaft and an outer peripheral surface in contact with an inner peripheral surface of the hub, and the reinforcing tube protrudes to a distal end side with respect to a distal end of the hub;
at least a part of an inner peripheral surface of the reinforcing tube in the hub is joined to at least a part of the outer peripheral surface of the shaft;
at least a part of an outer peripheral surface of the reinforcing tube in the hub is joined to at least a part of the inner peripheral surface of the hub;
a proximal end of the reinforcing tube is disposed on a proximal end side with respect to the distal end of the hub and on a distal end side with respect to the proximal end of the shaft; and
the outer peripheral surface of a shaft proximal portion of the shaft located on a proximal end side with respect to the proximal end of the reinforcing tube is joined to an inner peripheral surface of the hub.

17. The catheter according to claim 16, wherein a joined portion to which at least two of the shaft, the hub, or the reinforcing tube are joined is disposed at least at one location along an axial direction of the shaft.

18. The catheter according to claim 16, wherein at least a part of an outer peripheral surface of a shaft reinforcement target portion covered with the reinforcing tube of the shaft has an uneven surface formed with a surface roughness larger than a surface roughness of the outer peripheral surface of the shaft proximal portion located on a proximal end side with respect to the proximal end of the reinforcing tube of the shaft and brought into close contact with an inner peripheral surface of the reinforcing tube.

19. The catheter according to claim 16, wherein the reinforcing tube has a tubular non-welded portion slidable on an outer peripheral surface of the shaft in a range from the distal end to the proximal end side by a predetermined distance.

20. The catheter according to claim 16, wherein
at least a part of the shaft is formed of a material containing at least one of a polyamide resin or a polyamide elastomer and containing an X-ray contrast material and a pigment;
at least a part of the reinforcing tube is formed of a material containing at least one of a polyamide resin or a polyamide elastomer and containing the pigment; and
at least a part of the hub is formed of a material containing at least one of a polyamide resin, a polyurethane resin, a polyamide elastomer, or a polyurethane elastomer.
